(19) United States
(12) Patent Application Publication
(10) Pub. No.: US 2004/0070729 A1
(43) Pub. Date: Apr. 15, 2004

(54) Device and method for determining the viewing direction in terms of a fix reference co-ordinates system

(76) Inventors: Peter Wiebe, Gevelsberg (DE); Uwe Fakesch, Duesseldorf (DE); Oliver Nehrig, Moers (DE)

Correspondence Address: BEYER WEAVER & THOMAS LLP, P.O. BOX 778, BERKELEY, CA, 94704-0778, US

(21) Appl. No.: 10/465,934

(22) PCT Filed: Mar. 13, 2001
(86) PCT No.: PCT/EP01/02831

(30) Foreign Application Priority Data
Feb. 19, 2001 (DE) 10107685.1

(51) Int. Cl. A61B 3/14

(52) U.S. Cl. 351/209

(57) ABSTRACT

A device for determining the viewing direction relative to a fixed reference co-ordinate system comprises a detector for detecting electrooculograms so as to detect the viewing direction of the eyes of a user relative to the user's head. Furthermore, an inertial navigation system is provided for detecting the position of the head relative to said fixed reference co-ordinate system. Finally, the device comprises a computation unit for determining the viewing direction of the eyes of the user relative to said fixed reference co-ordinate system from the detected viewing direction of the eyes relative to the head and from the detected position of the head relative to said fixed reference co-ordinate system.

### BACKGROUND OF THE INVENTION

1. Field of the Invention

The present invention relates to devices and methods for determining the viewing direction and, in particular, it relates to devices and methods permitting a determination of an absolute viewing direction, i.e. a determination of a viewing direction relative to a fixed reference co-ordinate system.

2. Description of Prior Art

At the present time, which is increasingly dominated by technology and automation, numerous systems exist, which make use of an interaction between man and machine. All these systems need an interface between man as a user and the machine so as to be able to convey information between the user and the technical system. Typical examples of such interfaces in connection with a computer are keyboard, mouse, track ball and the like.

The above-mentioned interfaces serve to input information by hand. However, such interfaces can be problematic under various special conditions and physical limitations. For a user who executes text processing via a keyboard it may e.g. be cumbersome to move a cursor on the screen by means of a conventional mouse, since he has to remove one hand from the keyboard for this purpose. Furthermore, with computer-controlled machines and devices, one hand or both hands are often not free for carrying out inputs. In addition, the above-mentioned interfaces are problematic in the case of physically handicapped persons.

As has been described by P. Wiebe in: “Grundlage der Informationsübertragung mit Biosignalen für den Aufbau von technischen Kommunikationshilfen bei behinderten Menschen” (“Fundamental principles of information transmission with biosignals for constructing technical communication aids for physically handicapped persons”), Biomedical Engineering, Vol. 45, No. 1-2, 2000, pp. 14-19, man-machine interfaces are always based on acts that can be influenced intentionally and on biosignals, respectively. Such acts may consist e.g. of the generation of acoustic signals, electrophysiological signals and a movement of parts of the body, respectively.

Suggestions have already been made for man-machine interfaces, which permit an input of information into a system without hands or language being necessary for this purpose. There are e.g. systems in which a movement of the head is detected so as to cause on the basis of the movement of the head a movement of a cursor on a screen. Other systems use the viewing direction of the human eyes for controlling the respective man-machine interface. Information on systems of this kind and the fundamental technical principles of such systems can be gathered from the following literature sources:

LaCourse J. R., et al, “An Eye Movement Communication-Control System for the Disabled”, IEEE Transaction on Biomedical Engineering, 1990, 37(12), pp. 1215-1220;

Hutten H. et al, “Hochauflösendes Verfahren zur Kontrolle der Augenstellung” (“High-resolution method of controlling the position of the eyes”), Biomedizinische Technik, Vol. 43, supplementary volume 1, 1998, pp. 108 and 109;

G. Wieβpeiner et al, “Eye-Writer”, Biomedizinische Technik, Vol. 43, supplementary volume 2, 1998, pp. 158 to 161;

D. W. Pathmore et al, “Towards an EOG-Based Eye Tracker for Computer Control”, Third Annual ACM Conference on Assistive Technologies, 1998, pp. 197-203;

P. Wiebe et al, “Biosignalverarbeitung des menschlichen Elektrookulogramms (EOG) zur Steuerung von Computer-Eingabemedien für gelähmte Menschen” (“Biosignal processing of the human electrooculogram (EOG) for controlling computer input media for paralytics”) Biomedizinische Technik/Biomedical Engineering, Vol. 45, supplementary volume 1, 2000, pp. 184-185; and

D. R. Asche et al, “A Three-Electrode EOG for Use as a Communication Interface for the Non-Vocal, Physically Handicapped”, 29th ACEMB, Sheraton-Boston, Mass., Nov. 6 to 10, 1976, page 2.

All these known systems use the viewing direction of the eyes of a user relative to the head of the user, i.e. a relative eye movement. This eye movement can be detected because the eye produces an electric dipole field which is fixedly coupled to the eye and the eye movement, respectively, so that the eye movements will also change the position of the dipole field. An eye movement represents a biosignal, which can intentionally be reproduced and controlled to a large extent and which can be detected by measurement for moving on the basis thereof e.g. a cursor on a screen. The biosignal detected via the dipole field of the eye is referred to as electrooculogram (EOG).

An advantageous embodiment of a device for registering electrooculograms has been described in the above-mentioned publication “Biosignalverarbeitung des menschlichen Elektrookulogramms (EOG) zur Steuerung von Computer-Eingabemedien für gelähmte Menschen” by P. Wiebe. In the case of this embodiment a device is used, which can be worn by the user like a pair of spectacles, three electrodes being provided so as to be able to detect vertical movements of the eyes and horizontal movements of the eyes, i.e. the relative viewing direction of the user, as well as blinks. On the basis of the thus detected electrooculograms, man-machine interactions are then executed, e.g. a movement of a cursor on a screen or an input which, in the case of conventional interfaces, is carried out by means of a “mouse click” e.g. with the left mouse button of the computer.

The prior art additionally discloses sensors for inertial navigation systems by means of which it is possible to determine the position of an object in space. In this respect, reference is made e.g. to C. Lemair et al, “Surface Micromachined Sensors for Vehicle Navigation Systems”, Advance Microsystems for Automotive Application 98 (Berlin), Springer Verlag, 1998, pp. 112 to 133, and J. Söderkvist, “Micromachined Gyroscopes”, Sensors and Actuators A, 43, 1994, pp. 65 to 71.

### SUMMARY OF THE INVENTION

It is the object of the present invention to provide devices and methods which permit an interface between man and machine, which is flexible and convenient for the user.

According to a first aspect of the invention, this object is achieved by a device for determining the viewing direction relative to a fixed reference co-ordinate system, said device comprising:

a detector for detecting electrooculograms so as to detect the viewing direction of the eyes of a user relative to the user's head;

an inertial navigation system for detecting the position of the head relative to said fixed reference co-ordinate system; and

means for determining the viewing direction of the eyes of the user relative to said fixed reference co-ordinate system from the detected viewing direction relative to the head and from the detected position of the head relative to said fixed reference co-ordinate system.

According to a second aspect of the invention, the above object is achieved by a method of determining the viewing direction relative to a fixed reference co-ordinate system, said method comprising the following steps:

measuring the dipole field of the eyes of a user to detect the viewing direction of the eyes of the user relative to the head of the user;

detecting inertial signals so as to detect the position of the head of the user relative to the fixed reference co-ordinate system; and

determining the viewing direction of the eyes relative to the fixed reference co-ordinate system of the user from the detected viewing direction of the eyes of the user relative to the head of the user and from the detected position of the user relative to said fixed reference co-ordinate system.

It follows that the present invention provides methods and devices, which permit a determination of the viewing direction of human eyes relative to a fixed reference co-ordinate system; this viewing direction will be referred to as absolute viewing direction in the following, in contrast to the relative viewing direction of the eyes with respect to the user's head. The present invention is based on the finding that this absolute viewing direction depends on the relative position of the eyes with respect to the head as well as on the absolute head position, i.e. the head position relative to a fixed reference co-ordinate system. It follows that, for determining the absolute viewing direction, it is, on the one hand, necessary to detect the relative viewing direction; according to the present invention, this is preferably done by detecting an electrooculogram. On the other hand, it is necessary to detect the actual position of the head in the fixed reference co-ordinate system; according to the present invention, the position and the location, i.e. orientation, of the head in this fixed (absolute) co-ordinate system is, for this purpose, detected preferably by means of an inertial navigation system. The term inertial navigation system as used in connection with the present invention refers to systems which are capable of determining, normally on the basis of a defined initial condition, the head position relative to the fixed reference co-ordinate system. Head position means in this context the position in space of the head, or of a reference point thereof, as well as the orientation of the head relative to the fixed co-ordinate system. Hence, the present invention provides devices and methods for determining the absolute viewing direction with inertial and EOG signals.

In the most simple case, the inertial navigation system comprises means for detecting accelerations in at least three mutually perpendicular directions which correspond to the axes of a Cartesian co-ordinate system. Preferably, the inertial navigation system can additionally comprise means for detecting an inclination and a rotary speed about three mutually perpendicular axes, which can correspond to those of the Cartesian co-ordinate system. Any known system which is capable of determining the position, i.e. the spatial location and the orientation, of a body relative to a fixed reference can be used as an inertial navigation system according to the present invention.

When the viewing direction of the eyes relative to the user's head and the head position relative to the reference co-ordinate system are known, it is easily possible to determine therefrom, from a vectorial point of view, the viewing direction relative to the reference co-ordinate system, i.e. the absolute viewing direction of the eyes, making use of a suitable device, e.g. a microprocessor.

The thus determined absolute viewing direction can be used advantageously for a large number of applications. In VR applications (VR=Virtual Reality), for example, the control of scenes can be controlled in dependence upon the absolute viewing direction of the user wearing e.g. 3D spectacles. Furthermore, a man-machine interface can advantageously be realized by utilizing the detected absolute viewing direction, since e.g. a mouse pointer can easily be controlled precisely, depending on the point to which the user actually directs his view. According to the prior art, such a control is effected either in dependence upon only one movement of the eyes or only one movement of the head. The present invention allows in this connection a man-machine interface of increased flexibility and increased convenience for the user. The present invention can especially also be used in communication aids for physically handicapped persons.

In addition, the present invention can be used in field of ocular measurement techniques for medical purposes. The present invention can also be used in the field of motor vehicles or the like.

Further developments of the present invention are specified in the dependent claims.

### BRIEF DESCRIPTION OF THE DRAWINGS

In the following, preferred embodiments of the present invention will be explained in detail making reference to the drawings enclosed, in which

FIG. 1 shows a schematic representation for illustrating the relative viewing direction of the eyes and the absolute viewing direction of the eyes; and

FIG. 2 shows a preferred embodiment of the present invention used as a man-machine interface.

### DESCRIPTION OF PREFERRED EMBODIMENTS OF THE INVENTION

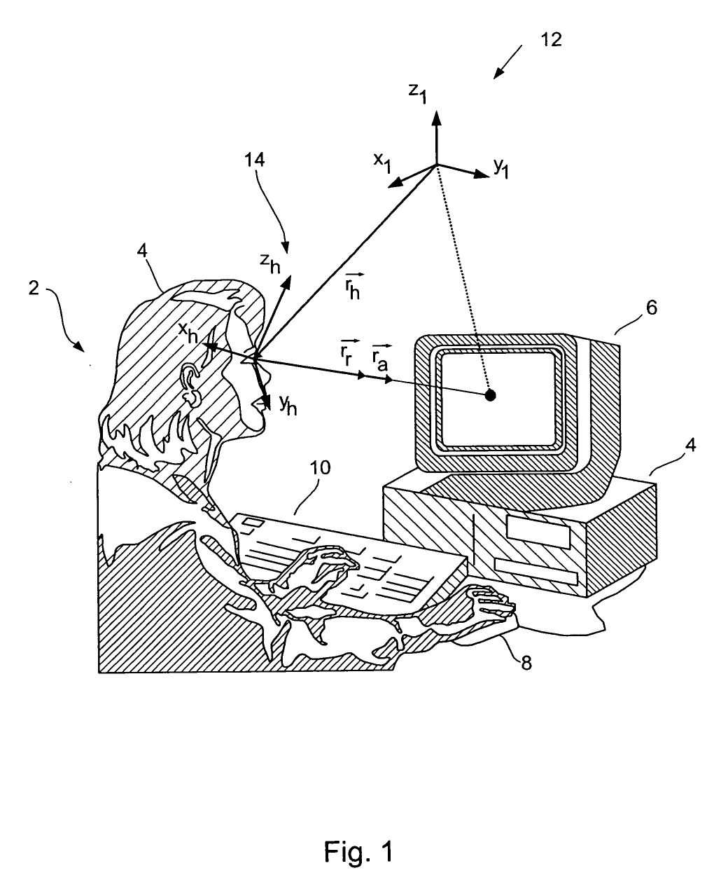

Making reference to FIG. 1, the connection between relative viewing direction, i.e. viewing direction of the eyes relative to the head of the user, and absolute viewing direction, i.e. viewing direction relative to a fixed reference co-ordinate system, will be explained in the following.

For this purpose, a user 2 is shown in FIG. 1, who sits in front of a computer 4 with an associated screen 6. Furthermore, the computer in FIG. 1 is equipped with a conventional man-machine interface in the form of a mouse 8 and a keyboard 10. FIG. 1 additionally shows a fixed reference co-ordinate system 12 with the co-ordinate axes xf, yf and zf and a co-ordinate system 14 which is associated with the head 4 of the user and which is therefore movable, said co-ordinate system 14 having three mutually perpendicular co-ordinate axes xh, zh and yh. The eyes of the user are concentrated on a point of fixation 16 on the screen 6. The resultant viewing direction of the user is indicated by the vector \overline{r}ᵣ in FIG. 1. The absolute viewing direction of the user 2 is additionally indicated by the vector \overline{r}ₐ in FIG. 1. \overline{r}ₐ as well as \overline{r}ᵣ have the same direction with respect to the fixed reference co-ordinate system 12. The viewing direction \overline{r}ᵣ, which is preferably determined on the basis of an electrooculogram according to the present invention, is here not related to the fixed reference co-ordinate system 12 but to the co-ordinate system 14 of the head 4 of the user 2. Furthermore, the absolute head position of the user 2 is indicated by the vector \overline{r}ₕ in FIG. 1, i.e. the position relative to the fixed reference co-ordinate system 12 or the origin thereof.

The position of the head co-ordinate system 14 relative to the fixed reference co-ordinate system 12 depends on the position of the head 4 relative to the reference co-ordinate system 12. It follows that, when the relative viewing direction \overline{r}ᵣ and the head position are detected, the absolute viewing direction, i.e. the viewing direction relative to the fixed coordinate system, can easily be determined from a vectorial point of view. Starting from the detected relative viewing direction, which is related to the head co-ordinate system 14, it will, for this purpose, suffice to take into account the position of the two co-ordinate systems 12 and 14 relative to one another, said position resulting from the head position relative to the fixed reference co-ordinate system. In this way, the user's absolute viewing direction, which is related to the fixed reference co-ordinate system 12, can easily be determined at any time, as long as the position of the head 4 relative to the fixed reference co-ordinate system is known. Position means in the present connection the spatial position of the head as well as the orientation, i.e. the inclination, rotation, etc. of the head relative to the reference co-ordinate system.

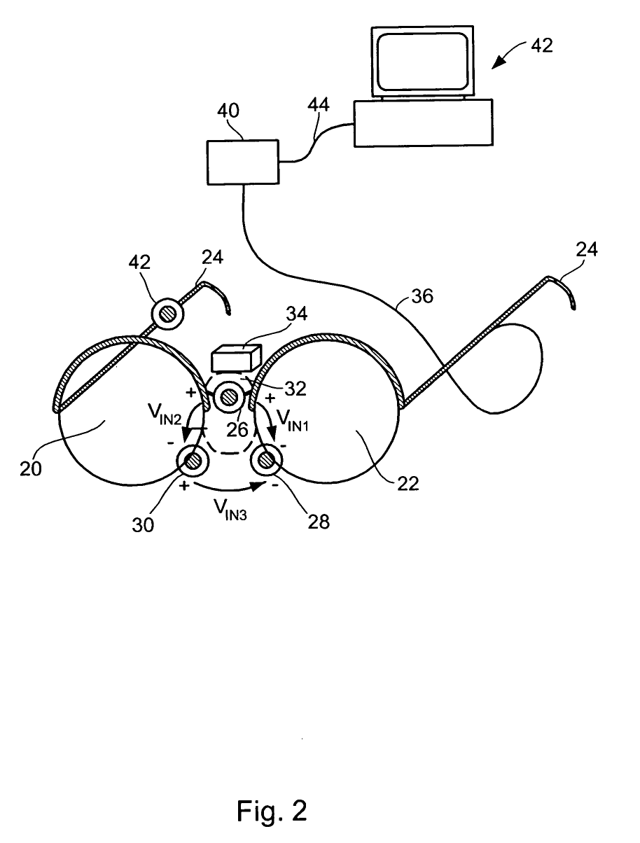

A preferred embodiment of a device according to the present invention used for determining the viewing direction relative to a fixed reference co-ordinate system, i.e. for determining the absolute viewing direction, is shown in FIG. 2 and can be referred to as EOG spectacles. This device has a configuration which corresponds essentially to that of known spectacles, but which need not have spectacle lenses. Optionally, spectacle lenses 20 and 22 can be provided for the right and for the left eye of a user, as indicated in FIG. 2, so as to compensate for visual defects of a user, if necessary.

The EOG spectacles can be worn like spectacles and comprise sidepieces 24 for this purpose. Furthermore, electrodes 26, 28, 30 are provided, by means of which the EOG spectacles rest on the nose of a user in the case of preferred embodiments. This means that the device is constructed such that a good contact between the skin and the electrodes, which is necessary for registering EOG signals, will already be guaranteed when the spectacles are put on. In addition, this will also guarantee that relative movements between the EOG spectacles and the head of the user will be avoided when the spectacles are worn. On the basis of an optional use of press-fastener contacts, also commercially available disposable electrodes can be used if hygienic measures should be necessary.

Via the electrodes, which rest on the surface of the user's skin, the biosignals produced by electric dipole fields can be tapped off by detecting by measurement the voltages VIN1, VIN2 and VIN3, which are shown in FIG. 2. For this purpose, the device is equipped with suitable preamplifiers, indicated in FIG. 2 by a broken line 32, so that the shortest possible lines to the preamplifiers can be realized, whereby interfering signal influences can be minimized and the signal quality improved.

With regard to this arrangement used for detecting the relative viewing direction, reference is made to the above-mentioned publication by P. Wiebe, “Biosignalverarbeitung des menschlichen Elektrookulogramms (EOG) zur Steuerung von Computer-Eingabemedien für gelähmte Menschen”.

The device according to the present invention shown in FIG. 2 additionally comprises a device for detecting the head position of the user, who wears the EOG spectacles, with respect to a fixed reference co-ordinate system; said device will be referred to as inertial navigation system in the following.

The tapped voltages VIN1, VIN2 and VIN3 as well as the output signals of the inertial navigation system 34, which can be preamplified in a suitable manner as well, are supplied via a line 36 to a processing means 40 in a suitable form, said processing means 40 determining on the basis of these signals the absolute viewing direction of the user who wears the EOG spectacles, i.e. the viewing direction relative to the fixed reference coordinate system. In addition, a ground electrode 42 is schematically shown on the sidepiece of the spectaclelike device representing the right sidepiece in FIG. 2; this ground electrode can, however, also occupy arbitrary other locations of the scalp surface.

When the functions of a conventional computer mouse are to be realized on the basis of the absolute viewing direction detected, the processing means 40 is coupled via a line 44 to a computer in a suitable form, said computer being schematically shown in FIG. 2 at 42. It goes without saying that the lines 36 and 44 shown in FIG. 2 can be replaced by wireless transmission mechanisms in a suitable manner.

In order to determine the relative viewing direction \overline{r}ᵣ (FIG. 1) of the user on the basis of the voltages VIN1, VIN2 and VIN3 tapped off via the electrodes 26, 28 and 30, the voltages are first preamplified, as has been stated hereinbefore, making use of suitable preamplifiers 32. The thus amplified detected voltages are transmitted to the processing means 40 in which averaging is optionally carried out on the basis of a respective predetermined number of sampled values so as to reduce the noise components. The signals obtained in this way on the basis of the voltages VIN1 and VIN2 are used as the basis for the vertical EOG in that a direction-related addition of these signals is preferably carried out, whereby the two horizontal components will compensate each other completely in the case of an exactly symmetrical dipole field distribution, so that the vertical EOG can be decoupled from the horizontal EOG to a large extent. This addition also has the effect that the amplitude of the vertical EOG will double. The signal used as horizontal EOG is the output signal obtained on the basis of the voltage VIN3.

The thus determined EOG signal shapes, which indicate the dipole field of the eyeball and, consequently, eyeball movements and the position of the eyeball relative to the head, can thus be used for determining the viewing direction of the eyes relative to the head.

The inertial navigation system 34 comprises, in the simplest version, one or a plurality of acceleration sensors which is or which are able to detect accelerations in the three directions of the axes of a Cartesian co-ordinate system. Preferably, the inertial navigation system also comprises inclination sensors and rotary speed sensors for detecting rotations and inclinations about the three axes. Starting from an initial condition, which is determined e.g. by using a suitable calibration of the inertial navigation system with respect to the reference co-ordinate system, and taking additionally into account the gravitational constant, the head position in space can be determined on the basis of the output signals of these sensors, since, on the on hand, the velocity v is the integral of the acceleration a over the time t and since, on the other hand, the position r is the integral of the velocity over the time.

The head position r can thus be determined from the detected accelerations a on the basis of the following equation:

\overline{r}ₕ=∫∫{\overline{a}ₓ(t)+\overline{a}_{y}(t)+\overline{a}_{z}(t)}dtdt

It follows that the head position can be determined in the manner known by means of appropriate calculations from the accelerations detected in the three directions of the Cartesian co-ordinate system. For carrying out these calculations and, in addition, for processing the EOG signals, a special hardware and/or software can be used.

The detected EOG signals and the output signals of the inertial navigation system can then be used for determining in the processing means 40 the absolute viewing direction, i.e. the viewing direction relative to a fixed co-ordinate system. The z-axis of the fixed co-ordinate system can, for example, be the vertical relative to the earth's surface, whereas the x-axis and the y-axis extend in the horizontal, i.e. they span the horizontal plane.

On the basis of the thus determined absolute viewing direction, suitable control signals for moving a cursor on the screen of the computer 42 can then be generated in the processing means 40. In addition, movements of the eyelid, which are also detected via the electrodes 26, 28 and 30, can be converted into respective input signals; in this connection, certain intentional movements of the eyelid can be interpreted e.g. as left mouse click or right mouse click. The output signals of the processing means 40 can in this case be prepared so as to be analogous to the output signals generated by a conventional mouse.

It follows that the present invention, which is used for detecting the absolute viewing direction, allows to provide a man-machine interface on the basis of the biosignals “relative viewing direction”, “movement of the eyelid” and “movement of the head”. The absolute viewing direction demanded in this respect is preferably determined by combining the obtainment of electrooculographic signals of the eyes and the position data of the head, which are determined by means of an inertial navigation system. In this way, numerous useful man-machine interfaces, which necessitate the absolute viewing direction of the user, can be realized.

While this invention has been described in terms of several preferred embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations, and equivalents as falling within the true spirit and scope of the present invention.

What is claimed is:

1. A device for determining the viewing direction relative to a fixed reference co-ordinate system, comprising:
a detector for detecting electoroculograms so as to detect the viewing direction of the eyes of a user relative to the user's head;
an inertial navigation system for detecting the position of the head relative to said fixed reference co-ordinate system; and
means for determining the viewing direction of the eyes of the user relative to said fixed reference co-ordinate system from the detected viewing direction relative to the head and from the detected position of the head relative to said fixed reference co-ordinate system.

2. A device according to claim 1, wherein the detector for detecting electrooculograms is arranged on a device that can be worn by a user like spectacles, and comprises at least three electrodes for detecting at least two voltages on the basis of eye dipole fields.

3. A device according to claim 1, wherein said inertial navigation system is arranged on the device that can be worn like spectacles.

4. A device according to claim 1, wherein the inertial navigation system comprises means for detecting accelerations in at least three mutually perpendicular directions.

5. A device according to claim 4, wherein the inertial navigation system additionally comprises means for detecting a rotation about at least three mutually perpendicular axes.

6. A method of determining the viewing direction relative to a fixed reference co-ordinate system, said method comprising the following steps:
measuring the dipole field of the eyes of a user so as to detect the viewing direction of the eyes of the user relative to the head of the user;
detecting inertial signals so as to detect the position of the head of the user relative to the fixed reference co-ordinate system; and
determining the viewing direction of the eyes of the user relative to the fixed reference co-ordinate system from the detected viewing direction of the eyes of the user relative to the head of the user and from the detected position of the user relative to said fixed reference co-ordinate system.

7. A method according to claim 6, wherein the inertial signals are detected on the basis of acceleration measurements and rotary speed measurements.
